(19) United States
(12) Patent Application Publication
(10) Pub. No.: US 2025/0025361 A1
(43) Pub. Date: Jan. 23, 2025

(54) MEDICAL SUPPORT DEVICE, MEDICAL COUCH APPARATUS, AND X-RAY DIAGNOSTIC APPARATUS

(71) Applicant: CANON MEDICAL SYSTEMS CORPORATION, Otawara-shi (JP)

(72) Inventors: Suzuna SAITO, Nasushiobara (JP); Yoshimasa KOBAYASHI, Nasushiobara (JP); Rie IKEZAKI, Sakura (JP); Hiroki TONOTSUKA, Otawara (JP); Katsuo TAKAHASHI, Utsunomiya (JP); Hayato KASAOKA, Nasushiobara (JP); Yoshinori SHIMIZU, Nasushiobara (JP); Seiichirou NAGAI, Otawara (JP)

(73) Assignee: CANON MEDICAL SYSTEMS CORPORATION, Otawara-shi (JP)

(21) Appl. No.: 18/777,252

(22) Filed: Jul. 18, 2024

(30) Foreign Application Priority Data
Jul. 20, 2023 (JP) 2023-118641

(51) Int. Cl. A61G 13/12; A61B 6/04; A61G 13/02

(52) U.S. Cl. CPC A61G 13/126; A61B 6/0407; A61B 6/0487; A61G 13/02; A61G 2210/50

(57) ABSTRACT

A medical support device according to an embodiment is configured to support a subject. The medical support device includes a movable part configured to operate in accordance with an operation inside the medical support device.

### CROSS REFERENCE TO RELATED APPLICATIONS

This application is based upon and claims the benefit of priority from the prior Japanese Patent Application No. 2023-118641, filed on Jul. 20, 2023, the entire contents of which are incorporated herein by reference.

### FIELD

Embodiments disclosed in the present specification and the drawings relate to a medical support device, a medical couch apparatus, and an x-ray diagnostic apparatus.

### BACKGROUND

In the related art, in X-ray photography, when a subject is not able to maintain a correct position and posture, the subject has been supported using a hand or an auxiliary tool of a medical radiologist (hereinafter, referred to as a technician).

However, when the subject is supported by the hand of the technician, the exposure dose of the technician may increase. In addition, there is a case where it is difficult to realize the support of the subject by the hand of the technician due to a labor shortage, and even if the support can be realized, the physical power of the technician may be wasted.

On the other hand, when the subject is supported by the auxiliary tool, in the related art, various sizes of auxiliary tools have been used or soft auxiliary tools such as foam materials have been used, but the subject may feel pain or move.

Therefore, in the related art, it has been difficult to simply and appropriately support the subject during X-ray photography.

### BRIEF DESCRIPTION OF THE DRAWINGS

FIG. 1 is a block diagram illustrating a configuration example of an X-ray diagnostic apparatus according to an embodiment.

FIG. 2 is a perspective view illustrating a configuration example of a medical support device according to the embodiment.

FIG. 3 is a side view illustrating a support tool of the medical support device according to the embodiment.

FIG. 4 is a front view illustrating the support tool of the medical support device according to the embodiment.

FIG. 5 is a diagram illustrating a movable part of the medical support device according to the embodiment.

FIG. 6 is a flowchart illustrating an operation example of the medical support device according to the embodiment.

FIG. 7 is a perspective view illustrating an operation example of the medical support device according to the embodiment.

FIG. 8 is a perspective view illustrating an operation example of the medical support device according to the embodiment subsequent to FIG. 7.

FIG. 9 is a plan view illustrating a medical support device according to a first modification of the embodiment.

FIG. 10 is a side view illustrating a medical support device according to a second modification of the embodiment.

FIG. 11 is a perspective view illustrating a medical support device according to a third modification of the embodiment.

FIG. 12 is a perspective view illustrating a medical support device according to a fourth modification of the embodiment.

FIG. 13 is a diagram illustrating a movable part of a medical support device according to a fifth modification of the embodiment.

FIG. 14 is a block diagram illustrating a configuration example of an X-ray diagnostic apparatus according to a sixth modification of the embodiment.

### DETAILED DESCRIPTION

Hereinafter, an embodiment of an X-ray diagnostic apparatus will be described with reference to the drawings. In the following description, an X-ray diagnostic apparatus having an over-tube type configuration in which an X-ray tube is located above a subject will be described as an example of the X-ray diagnostic apparatus, but the X-ray diagnostic apparatus may be configured as an under-tube type in which the X-ray tube is positioned below the subject. In the following description, components having substantially same functions and configurations are denoted by the same reference numerals, and redundant description will be made only when necessary.

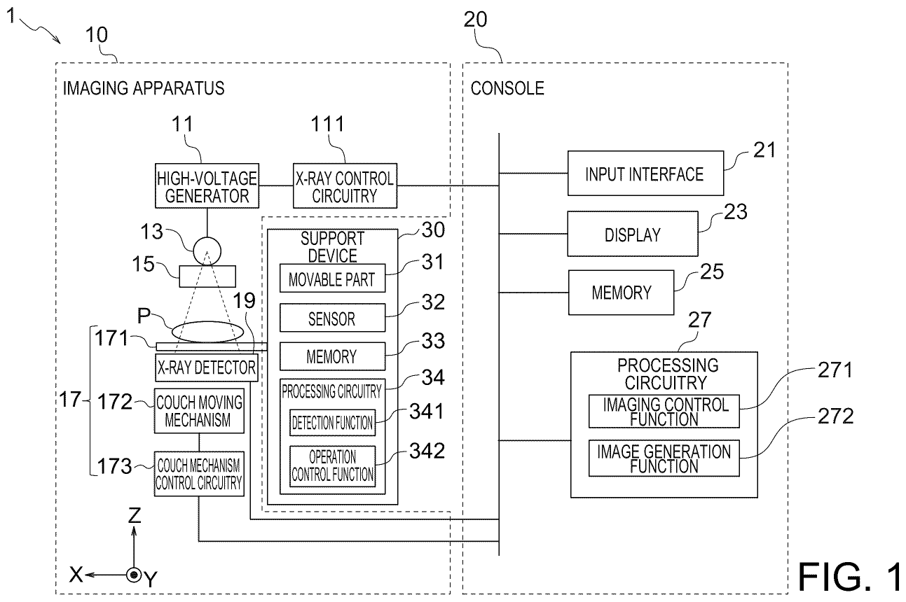

FIG. 1 is a diagram illustrating a configuration example of an X-ray diagnostic apparatus 1 according to the embodiment. As illustrated in FIG. 1, the X-ray diagnostic apparatus 1 according to the embodiment includes an imaging apparatus 10, a console 20, and a support device 30. The support device 30 is an example of a medical support device.

The imaging apparatus 10 is an apparatus that images a subject P using X-rays. The console 20 is, for example, a device for an operator to control the operation of the imaging apparatus 10 in an operation room that is a different room from an inspection room in which the imaging apparatus 10 is disposed. The imaging apparatus 10 and the console 20 are communicably connected. The support device 30 is a device that supports the subject P when imaging the subject P using X-rays. That is, the support device 30 is a device that supports the subject P positioned between an X-ray tube 13 and an X-ray detector 19 described later.

As illustrated in FIG. 1, the imaging apparatus 10 includes a high-voltage generator 11, the X-ray tube 13, an X-ray diaphragm 15, a couch apparatus 17, the X-ray detector 19, and an X-ray control circuitry 111. The X-ray tube 13 is an example of an X-ray generator. The couch apparatus 17 is an example of a medical couch apparatus. The couch apparatus 17 includes a top plate 171, a couch moving mechanism 172, and a couch mechanism control circuitry 173. The console 20 includes an input interface 21, a display 23, a memory 25, and a processing circuitry 27. In the following description, a direction along a longitudinal direction of the couch apparatus 17 is defined as a Y-axis direction, a direction along a lateral direction of the couch apparatus 17 is defined as an X-axis direction, and a direction orthogonal to the X-axis direction and the Y-axis direction is defined as a Z-axis direction.

The high-voltage generator 11 is a device that supplies a high voltage and a filament current to the X-ray tube 13. The high-voltage generator 11 includes an electric circuitry such as a transformer and a rectifier, and a high-voltage generating device. The high-voltage generating device has a function of generating a high voltage to be applied to the X-ray tube 13 and a filament current to be supplied to the X-ray tube 13. The X-ray control circuitry 111 controls the supply of the high voltage and the filament current by the high-voltage generating device. The high-voltage generating device may be a transformer type or an inverter type.

The X-ray tube 13 irradiates the subject P with X-rays. The X-ray tube 13 is a vacuum tube that generates X-rays by irradiating the anode (target) with thermal electrons from the cathode (filament) by application of a high voltage and supply of a filament current from the high-voltage generator 11. In the X-ray tube 13, X-rays are generated by thermal electrons colliding with the target. The X-ray tube 13 is, for example, a rotating anode type X-ray tube that generates X-rays by irradiating a rotating anode with thermal electrons. The type of the X-ray tube 13 is not limited to the rotating anode type, and any type can be applied to the X-ray tube 13. The X-ray diaphragm 15 narrows down the X-rays emitted from the X-ray tube 13. The X-ray diaphragm 15 is provided on a front surface of an X-ray emission window in the X-ray tube 13. The X-ray diaphragm 15 has, for example, four diaphragm blades made of a metal plate such as lead.

The X-ray tube 13 and the X-ray diaphragm 15 are movably supported by a stand (not illustrated) installed on a floor surface. The X-ray tube 13 and the X-ray diaphragm 15 can be rotated about a rotation shaft along the X-axis or moved along an upper surface of the top plate 171 by a moving mechanism (not illustrated) provided in the stand, for example.

The X-ray detector 19 detects the X-rays generated by the X-ray tube 13 and transmitted through the subject P. The X-ray detector 19 is, for example, an X-ray flat panel detector (hereinafter referred to as FPD). The FPD includes, for example, a plurality of semiconductor detection elements. The semiconductor detection element includes a direct conversion type in which X-rays are directly converted into electric signals and an indirect conversion type in which X-rays are converted into light by a phosphor and the light is converted into electric signals. Any type may be used for the FPD. Electric signals generated by the plurality of semiconductor detection elements in association with incidence of X-rays are output to an analog to digital converter (hereinafter referred to as A/D converter) (not illustrated). The A/D converter converts the electric signals into digital data. The A/D converter outputs the digital data to the processing circuitry 27. An image intensifier may be used as the X-ray detector 19.

The couch apparatus 17 is an apparatus for positioning the subject P. The couch apparatus 17 is moved by the couch moving mechanism 172 under the control of the couch mechanism control circuitry 173. For example, the couch moving mechanism 172 adjusts the reclining of the couch apparatus 17 by rotating the couch apparatus 17 about a rotation shaft along the X-axis direction. The couch moving mechanism 172 includes, for example, a drive source such as a motor provided in the stand described above, and a driving force transmission member that transmits a driving force of the drive source to the couch apparatus 17.

The input interface 21 receives input operations of various instructions and information from the operator. Specifically, the input interface 21 converts an input operation received from the operator into an electric signal and outputs the electric signal to the processing circuitry 27. For example, the input interface 21 is realized by a trackball, a switch button, a mouse, a keyboard, a touch pad that performs an input operation by touching an operation surface, a touch screen in which a display screen and the touch pad are integrated, a non-contact input circuitry using an optical sensor, a sound input circuitry, a foot switch, and the like. Note that the input interface 21 is not limited to an interface including physical operation components such as a mouse and a keyboard. For example, an electric signal processing circuitry that receives an electric signal corresponding to an input operation from an external input device provided separately from the device and outputs the electric signal to the control circuitry is also included in the example of the input interface 21.

The display 23 is a device that displays various types of information. For example, the display 23 converts image data transmitted from the processing circuitry 27 into an electric signal for display and outputs the electric signal. The display 23 is realized by a liquid crystal monitor, a cathode ray tube (CRT) monitor, a touch panel, and the like.

The memory 25 is a non-transitory storage device that stores various types of information, and is, for example, a hard disk drive (HDD), an optical disk, a solid state drive (SSD), an integrated circuitry storage device, or the like. The memory 25 stores, for example, a control program for controlling the X-ray diagnostic apparatus 1 and various types of data used for executing the control program. The memory 25 may be a drive device that reads and writes various types of information from and to a portable storage medium such as a compact disc (CD), a digital versatile disc (DVD), and a flash memory or a semiconductor memory element such as a random access memory (RAM), in addition to the HDD, the SSD, and the like.

The processing circuitry 27 is a circuitry that controls the operation of the entire X-ray diagnostic apparatus 1 according to the electric signal of the input operation input from the input interface 21. For example, the processing circuitry 27 includes an imaging control function 271 and an image generation function 272.

Here, for example, each processing function executed by the imaging control function 271 and the image generation function 272, which are components of the processing circuitry 27 illustrated in FIG. 1, is recorded in the memory 25 in the form of a program executable by a computer. The processing circuitry 27 is, for example, a processor. The processor constituting the processing circuitry 27 reads each program from the memory 25 and executes the program to realize a function corresponding to each read program. In other words, the processing circuitry 27 in a state of reading each program has each function illustrated in the processing circuitry 27 of FIG. 1.

Note that, in FIG. 1, a case where each processing function of the imaging control function 271 and the image generation function 272 is realized by a single processing circuitry 27 has been illustrated, but the embodiment is not limited thereto. For example, the processing circuitry 27 may be configured by combining a plurality of independent processors, and each processor may realize each processing function by executing each program. Furthermore, each processing function of the processing circuitry 27 may be realized by being appropriately distributed or integrated into a single or a plurality of processing circuitries.

The imaging control function 271 controls an imaging operation of the subject P by the imaging apparatus 10 based on, for example, an input operation received from the operator via the input interface 21. The imaging control function 271 controls the imaging operation of the subject P by controlling the X-ray control circuitry 111, the couch mechanism control circuitry 173, and the like. More specifically, the imaging control function 271 reads the control program stored in the memory 25, loads the control program on the memory in the processing circuitry 27, and controls each element of the X-ray diagnostic apparatus 1 according to the loaded control program.

The image generation function 272 generates image data based on the output from the X-ray detector 19. The image data is data of a medical image including a fluoroscopic image and a photographed image related to the subject P. The imaging control function 271 displays the generated image data on the display 23.

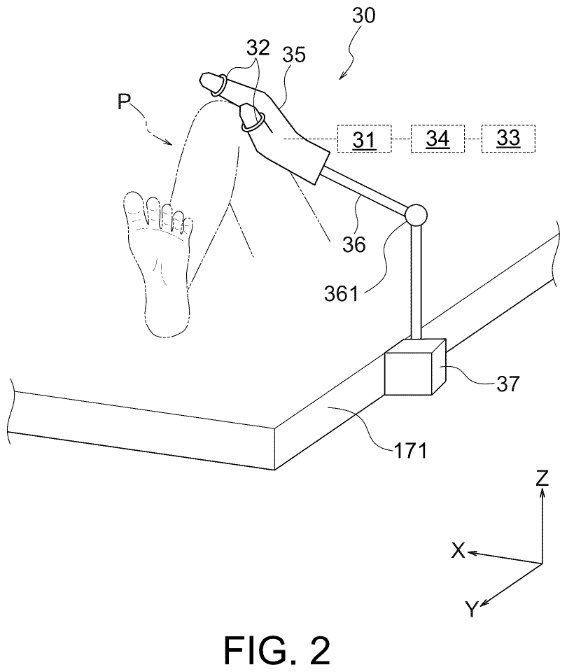

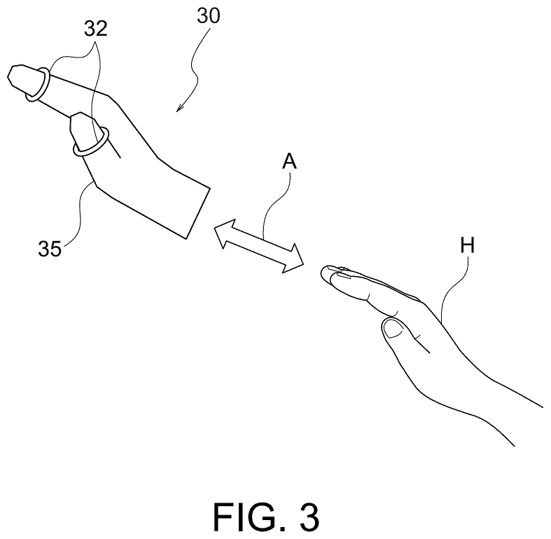

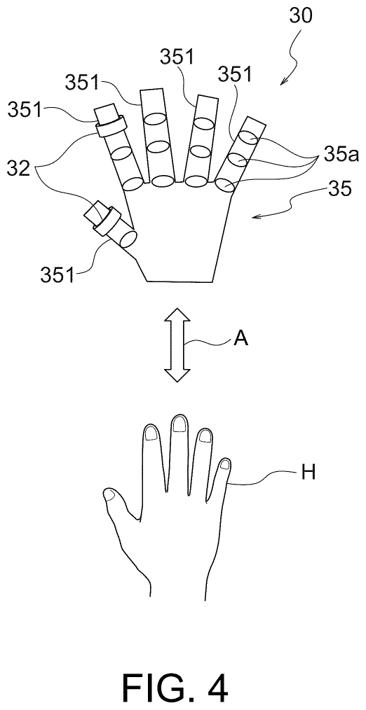

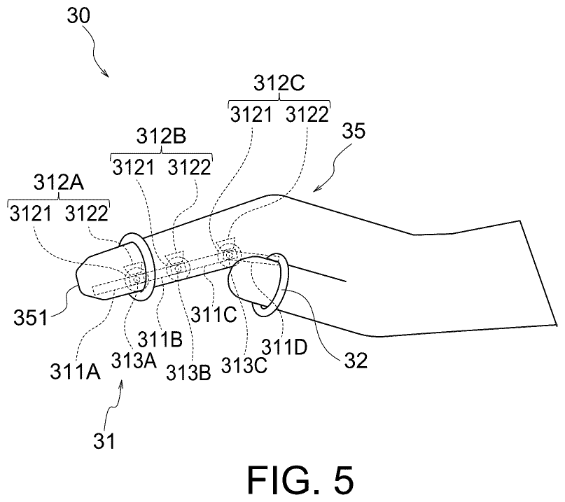

FIG. 2 is a perspective view illustrating a configuration example of the support device 30 according to the embodiment. FIG. 3 is a side view illustrating a support tool 35 of the support device 30 according to the embodiment. FIG. 4 is a front view illustrating the support tool 35 of the support device 30 according to the embodiment. FIG. 5 is a diagram illustrating a movable part 31 of the support device 30 according to the embodiment.

As illustrated in FIGS. 1 to 5, the support device 30 includes the movable part 31, a sensor 32, a memory 33, and a processing circuitry 34. The support device 30 further includes the support tool 35, an arm portion 36, and a fixer 37.

The support tool 35 has a shape into which a hand can be inserted. Arrows A in FIGS. 3 and 4 indicate an insertion direction and a detachment direction of a hand H with respect to the support tool 35. In the example illustrated in FIG. 4, the support tool 35 has a shape of a five-finger glove. That is, the support tool 35 has five branch portions 351 into which five fingers can be individually inserted. The support tool 35 is formed of, for example, a material having rigidity enough to maintain a shape into which a hand can be inserted and flexibility capable of bending a finger inside. The support tool 35 may be made of a resin material, a rubber material, or the like. Joint portions 35a provided at the positions corresponding to the joints of the finger on the branch portion 351 may be adjusted in rigidity and flexibility to be more easily bent than other portions of the branch portion 351.

As illustrated in FIG. 2, the support tool 35 is fixed to a distal end portion of the arm portion 36. The fixer 37 for fixing the support device 30 to the couch apparatus 17 is provided at a proximal end portion of the arm portion 36. The fixer 37 detachably fixes the support device 30 to the top plate 171 by a fixing method such as clamp fixing or screwing.

In the example illustrated in FIG. 2, a node 361 (that is, a rotation shaft) capable of changing a bending angle of the arm portion 36 is provided between the distal end portion and the proximal end portion of the arm portion 36. A posture of the support tool 35 can be changed by changing the bending angle of the arm portion 36. The node 361 of the arm portion 36 changes the bending angle of the arm portion 36, for example, in response to the operator moving the hand while the hand is inserted into the support tool 35. The node 361 of the arm portion 36 may be provided with a mechanical or electrical lock mechanism for maintaining the bending angle of the arm portion 36 after change.

The movable part 31 operates in accordance with an operation inside the support device 30. That is, the movable part 31 operates according to a manual operation in a state where the operator inserted a hand into the support tool 35. In the example illustrated in FIG. 5, the movable part 31 is provided inside the support tool 35.

In the example illustrated in FIG. 5, the movable part 31 includes a plurality of plate portions 311A, 311B, and 311C and a plurality of rotation regulators 312A, 312B, and 312C. The plurality of plate portions 311A, 311B, and 311C are arranged adjacent to each other along the finger. The plurality of plate portions 311A, 311B, and 311C are connected at positions corresponding to the joints of the fingers to be rotatable. The plurality of plate portions 311A, 311B, and 311C may be provided at positions in contact with pads of the fingers in the support tool 35. In the example illustrated in FIG. 5, the plurality of plate portions 311A, 311B, and 311C are provided along an index finger inside the branch portion 351 corresponding to the index finger. Similarly to the index finger, a plurality of plate portions are provided inside the branch portions 351 corresponding to fingers other than the index finger. However, the number of plate portions corresponding to the thumb is smaller by one than the number of plate portions corresponding to the other fingers.

In the example illustrated in FIG. 5, the plate portion 311A is provided along a distal phalanx at a position corresponding to the distal phalanx inside the support tool 35. The plate portion 311B is provided along a middle phalanx at a position corresponding to the middle phalanx inside the support tool 35. The plate portion 311C is provided along a proximal phalanx at a position corresponding to the proximal phalanx inside the support tool 35. The plate portion 311A is connected (that is, supported) to the plate portion 311B at a position corresponding to a distal inter phalangeal joint inside the support tool 35 to be rotatable about a rotation shaft 313A provided at a distal end portion of the plate portion 311B as a fulcrum. The plate portion 311B is connected to the plate portion 311C at a position corresponding to a proximal inter phalangeal joint inside the support tool 35 to be rotatable about a rotation shaft 313B provided at a distal end portion of the plate portion 311C as a fulcrum. The plate portion 311C is connected to a plate portion 311D to be rotatable about a rotation shaft 313C on the plate portion 311D provided corresponding to a metacarpal at a position corresponding to a metacarpal phalangeal joint inside the support tool 35. The plate portion 311D may be connected to a distal end portion of another plate portion to be rotatable.

The plurality of rotation regulators 312A, 312B, and 312C regulate rotation of each of the plurality of plate portions 311A, 311B, and 311C. The rotation regulator 312A is provided at a position corresponding to the rotation shaft 313A of the plate portion 311A, and regulates the rotation of the plate portion 311A. The rotation regulator 312B is provided at a position corresponding to the rotation shaft 313B of the plate portion 311B, and regulates the rotation of the plate portion 311B. The rotation regulator 312C is provided at a position corresponding to the rotation shaft 313C of the plate portion 311C, and regulates the rotation of the plate portion 311C. In the example illustrated in FIG. 5, the plurality of rotation regulators 312A, 312B, and 312C include a rotor portion 3121 that rotates integrally with the corresponding plate portions 311A, 311B, and 311C and a brake portion 3122 that regulates rotation of the rotor portion 3121. For example, the rotor portion 3121 may be a magnetic body, and the brake portion 3122 may be an electromagnetic brake that generates an electromagnetic force that attracts the rotor portion 3121 by energization. Such an electromagnetic brake that regulates the rotation of the plate portion can also be applied to a lock mechanism of the node 361 of the arm portion 36 described above.

The sensor 32 outputs a detection signal (that is, the electric signal) according to the presence of a hand. Specifically, the sensor 32 outputs the detection signal when the hand is inserted into the support tool 35, and does not output the detection signal when the hand is not inserted into the support tool 35. The sensor 32 outputs a detection signal according to a contact of a hand with the sensor 32 from inside of the support device 30. The contact of the hand with the sensor 32 may be direct contact or indirect contact via the support tool 35 or the like. The sensor 32 may output a detection signal according to the proximity of the hand to the sensor 32. At least two sensors 32 are provided corresponding to the thumb and a finger other than the thumb. In the example illustrated in FIGS. 2 and 3, the finger other than the thumb is the index finger. That is, the sensor 32 is provided at two locations of the branch portion 351 corresponding to the thumb and the branch portion 351 corresponding to the index finger. A specific mode of the sensor 32 is not particularly limited as long as the sensor 32 can output the detection signal according to the presence of the hand. The sensor 32 may be either a contact sensor or a non-contact sensor. The contact sensor may be, for example, a piezoelectric sensor or the like. The non-contact sensor may be, for example, a photosensor, a capacitance sensor, or the like.

The memory 33 of the support device 30 illustrated in FIG. 1 is a non-transitory storage device that stores various types of information, and is, for example, an HDD, an optical disk, an SSD, an integrated circuitry storage device, or the like. The memory 33 stores, for example, a control program for controlling the movable part 31 and various types of data used for executing the control program. The memory 33 may be a drive device that reads and writes various types of information from and to a portable storage medium such as a CD, a DVD, and a flash memory or a semiconductor memory element such as a RAM, in addition to the HDD, the SSD, and the like. For example, the memory 33 may be provided in the support tool 35, or may be provided in the fixer 37.

The processing circuitry 34 of the support device 30 is a circuitry that controls the operation of the movable part 31 according to an electric signal input from the sensor 32. For example, the processing circuitry 34 includes a detection function 341 and an operation control function 342. The detection function 341 is an example of a detector. The operation control function 342 is an example of an operation controller. For example, the processing circuitry 34 may be provided in the support tool 35 or may be provided in the fixer 37.

Here, for example, each processing function executed by the detection function 341 and the operation control function 342, which are components of the processing circuitry 34 illustrated in FIG. 1, is recorded in the memory 33 in the form of a program executable by a computer. The processing circuitry 34 is, for example, a processor. The processing circuitry 34 may include an electric circuitry other than the processor. The processor constituting the processing circuitry 34 reads each program from the memory 33 and executes the program to realize a function corresponding to each read program. In other words, the processing circuitry 34 in a state of reading each program has each function illustrated in the processing circuitry 34 of FIG. 1.

Note that, in FIG. 1, a case where each processing function of the detection function 341 and the operation control function 342 is realized by a single processing circuitry 34 has been illustrated, but the embodiment is not limited thereto. For example, the processing circuitry 34 may be configured by combining a plurality of independent processors, and each processor may realize each processing function by executing each program. Furthermore, each processing function of the processing circuitry 34 may be realized by being appropriately distributed or integrated into a single or a plurality of processing circuitries.

The detection function 341 detects the presence of a hand based on a detection signal output by the sensor 32. The operation control function 342 controls the operation of the movable part 31 to support the subject P according to the detection result of the presence of the hand by the detection function 341.

Specifically, when the hand is detected by the detection function 341, the operation control function 342 allows the operation of the movable part 31 according to the motion of the hand (that is, manual operation by the operator). On the other hand, when a hand is not detected by the detection function 341, the operation control function 342 causes the movable part 31 to support the subject P by regulating the operation of the movable part 31.

In the example illustrated in FIG. 5, when the hand is detected by the detection function 341, the operation control function 342 allows the operation of the movable part 31 by controlling the plurality of rotation regulators 312A, 312B, and 312C to not regulate the rotation of each of the plurality of plate portions 311A, 311B, and 311C. For example, the operation control function 342 does not generate the electromagnetic force that attracts the rotor portion 3121 corresponding to each brake portion 3122 by not energizing the plurality of brake portions 3122. On the other hand, when the hand is not detected by the detection function 341, the operation control function 342 regulates the operation of the movable part 31 by controlling the plurality of rotation regulators 312A, 312B, and 312C to regulate rotation of each of the plurality of plate portions 311A, 311B, and 311C. For example, the operation control function 342 generates the electromagnetic force that attracts the rotor portion 3121 corresponding to each brake portion 3122 by energizing the plurality of brake portions 3122.

In a case where the electromagnetic brake is provided at the node 361 of the arm portion 36, the operation control function 342 may control the electromagnetic brake to not regulate the rotation of the arm portion 36 when the hand is detected by the detection function 341. In addition, the operation control function 342 may control the electromagnetic brake to regulate the rotation of the arm portion 36 when the hand is not detected by the detection function 341.

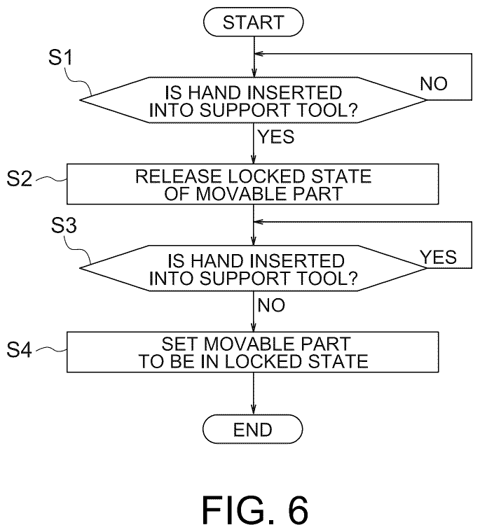

Next, an operation example of the support device 30 according to the embodiment configured as described above will be described. FIG. 6 is a flowchart illustrating an operation example of the support device 30 according to the embodiment. The flowchart of FIG. 6 is repeated as necessary. It is assumed that the operator's hand is not inserted into the support tool 35 in an initial state of FIG. 6. Therefore, in the initial state of FIG. 6, the sensor 32 does not output a detection signal, and the detection function 341 does not detect a hand. Therefore, the movable part 31 is in a locked state in which the operation is regulated by the operation control function 342. Then, from the initial state, first, as illustrated in FIG. 6, the detection function 341 determines whether the hand of the operator is inserted into the support tool 35, that is, whether the hand is present based on whether the detection signal is output from the sensor 32 (step S1).

When the operator's hand is inserted into the support tool 35 (step S1: YES), the operation control function 342 releases the locked state of the movable part 31 (step S2). Specifically, the operation control function 342 allows the operation of the movable part 31 by controlling the plurality of rotation regulators 312A, 312B, and 312C to not regulate the rotation of each of the plurality of plate portions 311A, 311B, and 311C. For example, the operation control function 342 does not generate the electromagnetic force that attracts the rotor portion 3121 to the brake portion 3122 by not energizing the brake portion 3122. On the other hand, when the operator's hand is not inserted into the support tool 35 (step S1: NO), the detection function 341 repeats the determination as to whether the operator's hand is inserted into the support tool 35 (step S1).

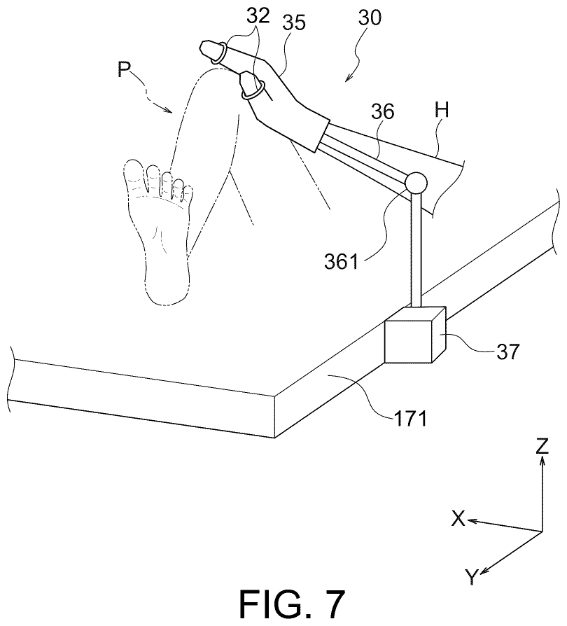

FIG. 7 is a perspective view illustrating an operation example of the support device 30 according to the embodiment. As illustrated in FIG. 7, in a state where a hand H of the operator is inserted into the support tool 35, the locked state of the movable part 31 is released, so that the operator can adjust the bending amount of the finger in the support tool 35. Since the bending amount of the finger can be adjusted, a support force (that is, gripping force) for supporting a support target portion (for example, a knee in FIG. 7) of the subject P can be adjusted via the support tool 35.

After the locked state of the movable part 31 is released, as illustrated in FIG. 6, the detection function 341 determines whether the hand of the operator is inserted into the support tool 35, based on whether the detection signal is output from the sensor 32 (step S3).

When the operator's hand is not inserted into the support tool 35 (step S3: NO), the operation control function 342 sets the movable part 31 in the locked state (step S4). Specifically, the operation control function 342 regulates the operation of the movable part 31 by controlling the plurality of rotation regulators 312A, 312B, and 312C to regulate the rotation of each of the plurality of plate portions 311A, 311B, and 311C. For example, the operation control function 342 generates the electromagnetic force that attracts the rotor portion 3121 to the brake portion 3122 by energizing the brake portion 3122. On the other hand, when the operator's hand is inserted into the support tool 35 (step S3: YES), the detection function 341 repeats the determination as to whether the operator's hand is inserted into the support tool 35 (step S3).

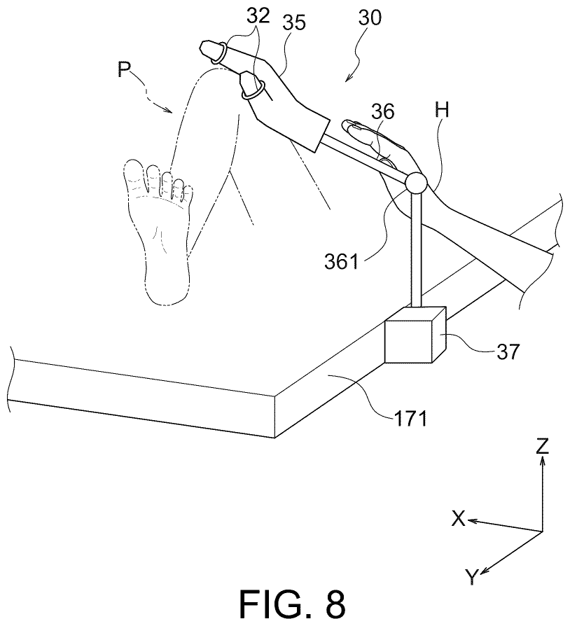

FIG. 8 is a perspective view illustrating an operation example of the support device 30 according to the embodiment subsequent to FIG. 7. As illustrated in FIG. 8, in the locked state, the movable part 31 holds an operation state (for example, rotation angles of the plate portions 311A, 311B, and 311C) immediately before the hand H of the operator is removed from the support tool 35. Therefore, the movable part 31 can maintain the support force of the support target portion of the subject P adjusted when the operator inserted the hand H into the support tool 35. As a result, the support device 30 can appropriately support the support target portion of the subject P even after the operator removes the hand H from the support tool 35.

As described above, in the support device 30 according to the embodiment, the movable part 31 operates according to the operation inside the support device 30.

As a result, when the operation inside the support device 30 is performed, the support force of the subject P can be intuitively adjusted by the operation of the movable part 31 according to the operation, and when the operation is not performed, the operation of the movable part 31 can be regulated to maintain the support force. Therefore, an appropriate support force can be applied to the subject P by intuitively adjusting the support force by a simple operation inside the support device 30. As a result, the subject P can be easily and appropriately supported. Furthermore, an imaging position of the subject P can be appropriately held by appropriately supporting the subject P. By appropriately holding the imaging position of the subject P, the subject P can be appropriately imaged. In addition, since the operator does not need to continuously support the subject P during imaging, the exposure dose can be reduced and the labor of the operator can be reduced. In addition, since the support and imaging of the subject P can be performed by one operator, it is also possible to resolve a labor shortage.

Furthermore, in the support device 30 according to the embodiment, the sensor 32 outputs a detection signal according to the presence of the hand H. In addition, the detection function 341 detects the presence of a hand H based on a detection signal output by the sensor 32. In addition, the operation control function 342 controls the operation of the movable part 31 to support the subject P according to the detection result of the presence of the hand H by the detection function 341.

As a result, it is possible to easily and appropriately grasp whether the operation inside the support device 30 is performed based on the detection signal of the sensor 32 and to control the operation of the movable part 31, and thus, it is possible to more easily and appropriately support the subject P.

Furthermore, in the support device 30 according to the embodiment, when the hand H is detected by the detection function 341, the operation control function 342 allows the operation of the movable part 31 according to the motion of the hand H. In addition, when the hand H is not detected by the detection function 341, the operation control function 342 causes the movable part 31 to support the subject P by regulating the operation of the movable part 31.

As a result, it is possible to easily and appropriately switch the adjustment of the support force by the operator and the maintaining of the support force by the movable part 31 based on the detection result of the detection function 341, and thus, it is possible to more easily and appropriately support the subject P.

In the support device 30 according to the embodiment, the movable part 31 includes the plurality of plate portions 311A, 311B, and 311C and the plurality of rotation regulators 312A, 312B, and 312C. The plurality of plate portions 311A, 311B, and 311C are arranged adjacent to each other along the finger, and are connected at positions corresponding to the joints of the finger to be rotatable. The plurality of rotation regulators 312A, 312B, and 312C regulate rotation of each of the plurality of plate portions 311A, 311B, and 311C. When the hand H is detected by the detection function 341, the operation control function 342 allows the operation of the movable part 31 by controlling the plurality of rotation regulators 312A, 312B, and 312C to not regulate the rotation of each of the plurality of plate portions 311A, 311B, and 311C. In addition, when the hand H is not detected by the detection function 341, the operation control function 342 regulates the operation of the movable part 31 by controlling the plurality of rotation regulators 312A, 312B, and 312C to regulate rotation of each of the plurality of plate portions 311A, 311B, and 311C.

As a result, when the hand H is detected by the detection function 341, the rotation of the plurality of plate portions 311A, 311B, and 311C along the finger is not regulated, so that the operator can easily and more intuitively adjust the support force. Further, when the hand H is not detected by the detection function 341, the rotation of the plurality of plate portions 311A, 311B, and 311C is regulated, so that the movable part 31 can easily and appropriately maintain the support force adjusted by the operator. Therefore, the subject P can be more easily and appropriately supported.

Furthermore, in the support device 30 according to the embodiment, the sensor 32 outputs a detection signal in response to contact of a hand from the inside of the support device 30.

As a result, the presence of the hand can be easily and appropriately detected, so that the subject P can be more easily and appropriately supported.

Furthermore, in the support device 30 according to the embodiment, at least two sensors 32 are provided corresponding to the thumb and a finger other than the thumb.

As a result, since the presence of the hand can be appropriately detected by the at least two sensors 32 provided at appropriate positions, the subject P can be more appropriately supported.

Further, in the support device 30 according to the embodiment, a finger other than the thumb is the index finger.

As a result, by providing the sensor 32 at a position corresponding to the main thumb and index finger to support the subject P, it is possible to more appropriately perform switching between adjustment of the support force by the operator and maintaining of the support force by the movable part 31. Therefore, the subject P can be more appropriately supported.

Further, in the support device 30 according to the embodiment, the support device 30 further includes the support tool 35 having a shape into which a hand can be inserted.

As a result, since the operator can support the subject P through the support tool 35 in a state where the hand is inserted into the support tool 35, the support force can be adjusted more intuitively. Therefore, the subject P can be more easily and appropriately supported.

Further, in the support device 30 according to the embodiment, the movable part 31 is provided inside the support tool 35.

As a result, the operator can allow the movable part 31 to operate according to the motion of the finger in a state where the hand is inserted into the support tool 35, so that the support force can be adjusted more intuitively. Therefore, the subject P can be more easily and appropriately supported.

In addition, the support device 30 according to the embodiment is fixed to the top plate 171 of the couch apparatus 17.

As a result, the subject P can be appropriately supported at the time of imaging in a lying state.

Various modifications described below can be applied to the embodiment.

### (First Modification)

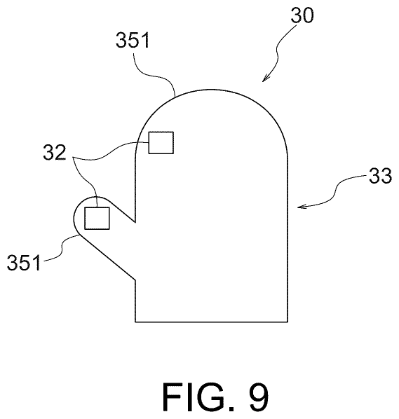

First, a first modification in which the shape of the support tool 35 is different will be described. FIG. 9 is a plan view illustrating a support device 30 according to the first modification of the embodiment. In the above-described embodiment, an example of the support tool 35 having the shape of a glove with five fingers has been described. On the other hand, in the example illustrated in FIG. 9, a support tool 35 has a mitten shape. That is, the support tool 35 has two branch portions 351 corresponding to the thumb and the fingers other than the thumb. Two sensors 32 are provided corresponding to the two branch portions 351.

According to the first modification, since the support tool 35 has the shape of a mitten, it is possible to facilitate insertion and removal of a hand into and from the support tool 35.

### (Second Modification)

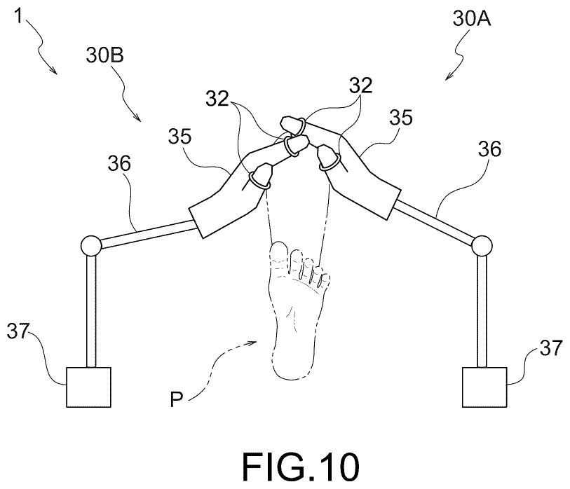

Next, a second modification in which the subject P is supported by both hands will be described. FIG. 10 is a side view illustrating a support device according to the second modification of the embodiment. As illustrated in FIG. 10, the X-ray diagnostic apparatus 1 according to the second modification includes two types of support devices 30A and 30B, that is, a right-handed support device 30A and a left-handed support device 30B to be able to maintain the support force of the subject P with both hands of the operator.

The right-handed support device 30A is the same as the configuration illustrated in FIG. 2 and the like. The left-handed support device 30B has a mirror-symmetrical shape with respect to the right-handed support device 30A. Note that the right-handed support tool 35 and component portions 31 and 32 provided in the support tool 35 and the left-handed support tool 35 and the components 31 and 32 provided in the support tool 35 may share a part of the fixer 37 and the arm portion 36.

According to the second modification, the support force of the right hand of the operator can be maintained by regulating the motion of the movable part 31 of the right-handed support device 30A. Further, the support force of the left hand of the operator can be maintained by regulating the motion of the movable part 31 of the left-handed support device 30B. As a result, the subject P can be more appropriately supported.

### (Third Modification)

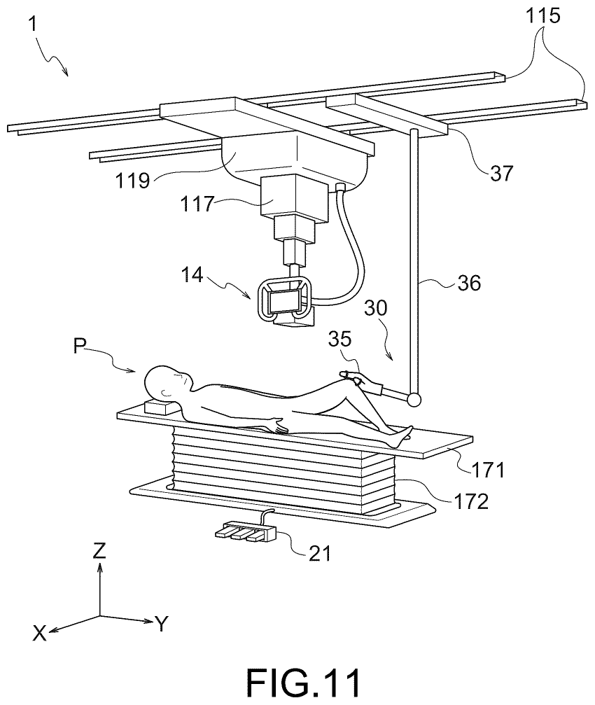

Next, a third modification in which the support device 30 is fixed to a ceiling will be described. FIG. 11 is a perspective view illustrating a support device 30 according to the third modification of the embodiment. In the above-described embodiment, an example of the support device 30 fixed to the top plate 171 of the couch apparatus 17 has been described. On the other hand, in the example illustrated in FIG. 11, the support device 30 is fixed to the ceiling.

Specifically, the fixer 37 of the support device 30 is supported by a rail 115 provided on the ceiling to be movable along a Y-axis direction which is a longitudinal direction of the rail 115. The support tool 35 and component portions 31 and 32 (not illustrated) provided in the support tool 35 are suspended from the rail 115 via the fixer 37 and the arm portion 36. An operation box 14 including the X-ray tube 13 and the X-ray diaphragm 15 is suspended from the rail 115 via a support portion 119 supported to be able to slide along the rail 115 and a support portion 117 extending downward from the support portion 119. The operator can freely set the distance and direction to the X-ray detector 19 by operating the operation box 14.

According to the third modification, since the support device 30 can be fixed to the ceiling, the degree of freedom in installation of the support device 30 can be improved.

### (Fourth Modification)

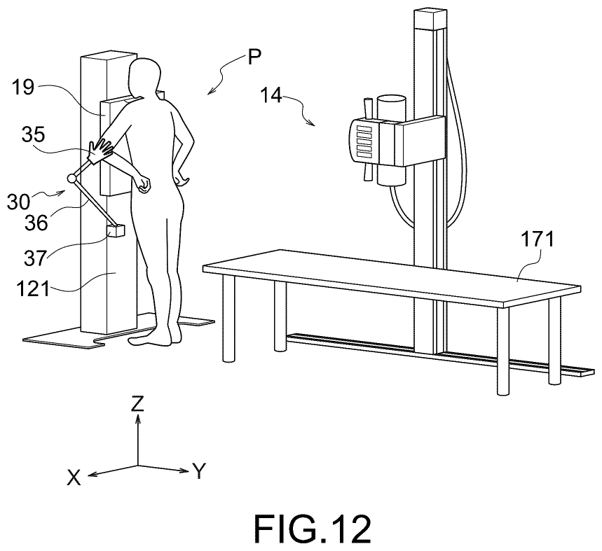

Next, a fourth modification in which the subject P is supported at the time of imaging in an upright state will be described. FIG. 12 is a perspective view illustrating a support device 30 according to the fourth modification of the embodiment. In the above-described embodiment, an example of the support device 30 that supports the subject P at the time of imaging in the lying state has been described. On the other hand, in the example illustrated in FIG. 12, the support device 30 supports the subject P at the time of imaging in the upright state.

Specifically, the support device 30 is supported by a stand 121 installed on a floor surface. The stand 121 supports the X-ray detector 19 in a direction orthogonal to the floor surface. An orientation of the operation box 14 is adjusted to an orientation in which the subject P positioned in front of the X-ray detector 19 is irradiated with X-rays. The support device 30 supports the stand 121, that is, the subject P positioned in front of the X-ray detector 19. In the example illustrated in FIG. 12, the support device 30 supports an elbow of the subject P.

According to the fourth modification, since the subject P can be supported at the time of imaging in the standing state, convenience and versatility of the support device 30 can be improved.

### (Fifth Modification)

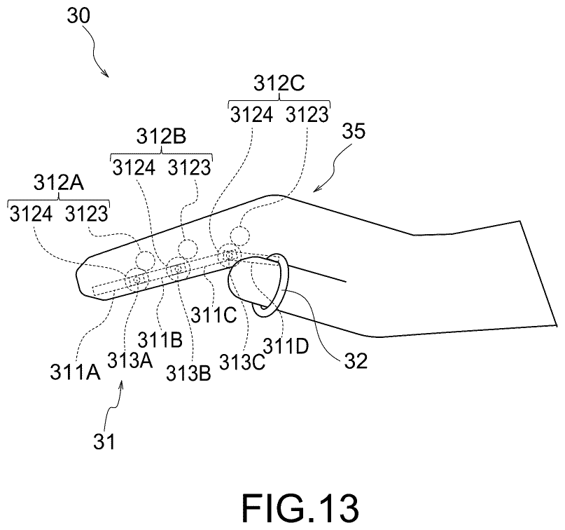

Next, a modification of the movable part 31 will be described. FIG. 13 is a diagram illustrating a movable part 31 of a support device 30 according to a fifth modification of the embodiment. In the above-described embodiment, an example has been described in which the plurality of rotation regulators 312A, 312B, and 312C of the movable part 31 include the rotor portion 3121 that rotates integrally with the corresponding plate portions 311A, 311B, and 311C and the brake portion 3122 that regulates the rotation of the rotor portion 3121. On the other hand, in the example illustrated in FIG. 13, the plurality of rotation regulators 312A, 312B, and 312C include a motor 3123 and a clutch portion 3124 that connects or disconnects the motor 3123 to or from the corresponding rotation shafts 313A, 313B, and 313C. The clutch portion 3124 is, for example, an electromagnetic clutch.

When a hand is detected by the detection function 341, the operation control function 342 does not energize the plurality of clutch portions 3124, thereby causing each clutch portion 3124 to disconnect the motor 3123 and the rotation shafts 313A, 313B, and 313C. As a result, since the rotation of each of the plate portions 311A, 311B, and 311C with the rotation shafts 313A, 313B, and 313C as fulcrums is not regulated, the operator can adjust the support force of the subject P by moving the fingers.

On the other hand, when a hand is not detected by the detection function 341, the operation control function 342 energizes the plurality of clutch portions 3124, thereby causing each clutch portion 3124 to connect the motor 3123 and the rotation shafts 313A, 313B, and 313C. In a state where the motor 3123 and the rotation shafts 313A, 313B, and 313C are connected, the operation control function 342 performs energization control of the motor 3123 so that the motor 3123 is set to be in a locked state. As a result, since the rotation of each of the plurality of plate portions 311A, 311B, and 311C is regulated, the movable part 31 can maintain the support force adjusted by the operator. Note that, in a case where the hand is not detected by the detection function 341, the operation control function 342 can also rotationally drive the motor 3123 in a direction of clenching the hand to apply an appropriate torque to the subject P.

According to the fifth modification, the degree of freedom in designing the movable part 31 can be improved.

### (Sixth Modification)

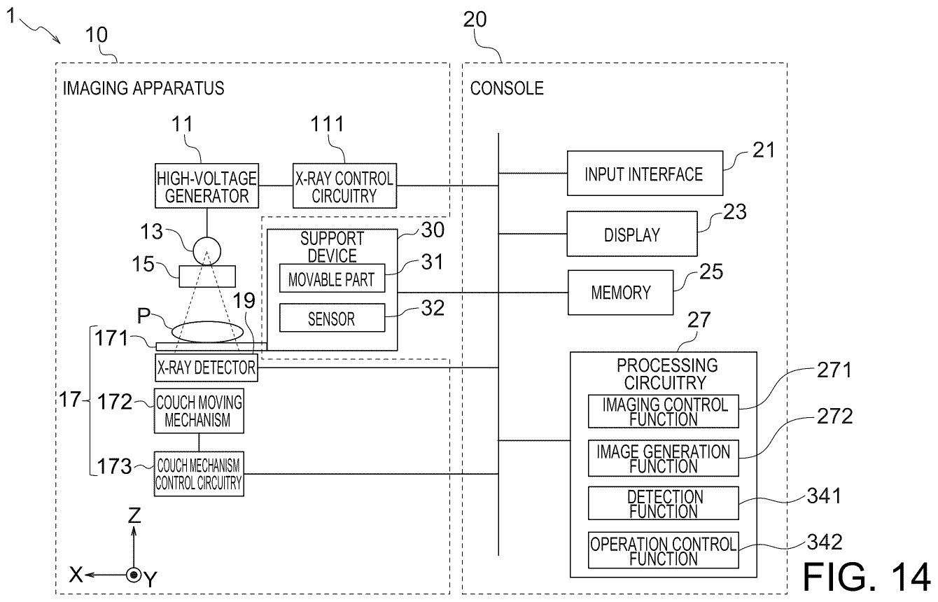

FIG. 14 is a block diagram illustrating a configuration example of an X-ray diagnostic apparatus 1 according to a sixth modification of the embodiment. In the embodiment described above, the support device 30 independently includes the memory 33 and the processing circuitry 34. In contrast, as illustrated in FIG. 14, the memory 33 and the processing circuitry 34 of the support device 30 may be integrated with the memory 25 and the processing circuitry 27 of the console 20. That is, the detection function 341 and the operation control function 342 may be included in the processing circuitry 27 of the console 20. The operation control function 342 may remotely control the operation of the movable part 31 based on an electric signal of an input operation from the input interface 21 in addition to the detection vibration of the sensor 32.

Note that the term “processor” used in the above description means, for example, a central processing unit, a graphics processing unit, or a circuitry such as an application specific integrated circuitry or a programmable logic device (for example, a simple programmable logic device, a complex programmable logic device, and a field programmable gate array). A processor realizes a function by reading and executing a program stored in a memory. Instead of storing the program in the memory, the program may be directly incorporated in a circuitry of the processor. In this case, the processor realizes the function by reading and executing the program incorporated in the circuitry. Note that the processor is not limited to a case of being configured as a single processor circuitry, and a plurality of independent circuitries may be combined to be configured as one processor to realize the function. Furthermore, a plurality of components in FIG. 1 may be integrated into one processor to realize the function.

According to at least one embodiment described above, the subject can be easily and appropriately supported.

Although several embodiments have been described above, the embodiments have been presented only as examples, and are not intended to limit the scope of the invention. The novel devices and methods described herein can be realized in a variety of other forms. In addition, various omissions, substitutions, and changes can be made to the forms of the apparatus and the method described in the present specification without departing from the gist of the invention. The appended claims and their equivalents are intended to include such forms and modifications as to fall within the scope and spirit of the invention.

What is claimed is:

1. A medical support device configured to support a subject, the device comprising:
a movable part configured to operate in accordance with an operation inside the medical support device.

2. The medical support device according to claim 1, further comprising:
at least one sensor configured to output a detection signal according to presence of a hand; and
processing circuitry configured to detect the presence of the hand based on the detection signal output by the sensor, and
control an operation of the movable part to support the subject according to a detection result of the presence of the hand.

3. The medical support device according to claim 2, wherein the processing circuitry is configured to
allow the operation of the movable part according to a motion of the hand when the hand is detected, and
regulate the operation of the movable part to cause the movable part to support the subject when the hand is not detected.

4. The medical support device according to claim 3,
wherein the movable part comprises
a plurality of plate portions arranged adjacent to each other along a finger, the plurality of plate portions being connected to each other at positions corresponding to joints of the finger to be rotatable; and
a plurality of rotation regulators configured to regulate rotation of each of the plurality of plate portions, and
wherein the processing circuitry is configured to
control the plurality of rotation regulators to not regulate the rotation of each of the plurality of plate portions when the hand is detected to allow the operation of the movable part, and
control the plurality of rotation regulators to regulate the rotation of each of the plurality of plate portions when the hand is not detected to regulate the operation of the movable part.

5. The medical support device according to claim 2, wherein the sensor is configured to output the detection signal according to contact of the hand from inside the medical support device.

6. The medical support device according to claim 2, wherein at least two of the sensors are provided corresponding to a thumb and a finger other than the thumb.

7. The medical support device according to claim 6, wherein the finger other than the thumb is an index finger.

8. The medical support device according to claim 1, further comprising: a support tool having a shape into which a hand is able to be inserted.

9. The medical support device according to claim 8, wherein the support tool has five branch portions into which five fingers are able to be individually inserted.

10. The medical support device according to claim 8, wherein the support tool comprises two branch portions corresponding to a thumb and fingers other than the thumb.

11. The medical support device according to claim 8, wherein the movable part is provided inside the support tool.

12. The medical support device according to claim 4, wherein the rotation regulator comprises a rotor portion configured to rotate integrally with the plate portion and a brake portion configured to regulate rotation of the rotor portion.

13. The medical support device according to claim 4,
wherein the rotation regulator comprises a motor and a clutch portion configured to connect or disconnect the motor to or from a rotation shaft of the plate portion, and
the processing circuitry is configured to regulate the operation of the movable part by controlling the motor to be in a locked state in a state where the motor is connected to the rotation shaft.

14. The medical support device according to claim 8, further comprising:
an arm portion in which the support tool is fixed to a distal end portion, the arm portion comprising a node configured to be capable of changing a bending angle of the arm portion; and
a fixer provided at a proximal end portion of the arm portion and configured to fix the medical support device.

15. A medical couch apparatus comprising:
a top plate;
a medical support device configured to support a subject on the top plate,
wherein the medical support device comprises a movable part configured to operate in accordance with an operation inside the medical support device.

16. The medical couch apparatus according to claim 15, wherein the medical support device is fixed to the top plate.

17. An X-ray diagnostic apparatus comprising:
an X-ray generator configured to generate X-rays;
an X-ray detector configured to detect X-rays generated by the X-ray generator and passing through a subject; and
a medical support device configured to support the subject positioned between the X-ray generator and the X-ray detector,
wherein the medical support device comprises a movable part configured to operate in accordance with an operation inside the medical support device.

18. The X-ray diagnostic apparatus according to claim 17, further comprising: a medical couch apparatus comprising a top plate,
wherein the X-ray generator and the medical support device are supported by a ceiling, and
the medical support device is configured to support the subject positioned on the top plate.

19. The X-ray diagnostic apparatus according to claim 18, wherein the medical support device comprises a left-handed medical support device and a right-handed medical support device.

20. The X-ray diagnostic apparatus according to claim 17,
wherein the X-ray detector and the medical support device are supported by a stand installed on a floor surface, and
the medical support device is configured to support the subject positioned in front of the stand.
